(19) United States
(12) Patent Application Publication
(10) Pub. No.: US 2006/0029640 A1
(43) Pub. Date: Feb. 9, 2006

(54) Medical devices with surface modification for regulating cell growth on or near the surface

(76) Inventors: Jeremy L. Gilbert, Fayetteville, NY (US); Naim Istephanous, Roseville, MN (US); Darrel F. Untereker, Oak Grove, MN (US); Kenneth E. Rohly, Lino Lakes, MN (US)

Correspondence Address: MEDTRONIC, INC., 710 MEDTRONIC PARK, MINNEAPOLIS, MN, 55432-9924, US

(21) Appl. No.: 11/198,090

(22) Filed: Aug. 5, 2005

Related U.S. Application Data
(60) Provisional application No. 60/599,261, filed on Aug. 5, 2004.

(51) Int. Cl. A61F 2/82; A61F 2/02

(52) U.S. Cl. 424/424; 623/1.44; 623/1.42

(57) ABSTRACT

High oxidation state metal-containing compounds are formed on or applied to the surfaces of medical devices. These compounds can cause cytostatic, cytotoxic, and anti-proliferative effects to the cells on or near the implanted medical devices and thus reduce or eliminate undesirable cell growth or accumulation on the medical devices.

### FIELD OF THE INVENTION

This invention relates to medical devices with surface modification for reducing or eliminating undesirable cell growth or accumulation as a result of implantation of the devices. More particularly, the surfaces of the medical devices are modified to include high oxidation state metal-containing compounds that produce desired cytostatic, cytotoxic, and anti-proliferative effects when the medical devices are implanted. The invention further relates to methods for treating proliferative diseases in mammals by implantation of medical devices with surface modification for reducing or eliminating undesirable cell growth.

### BACKGROUND OF THE INVENTION

Implantation of medical devices to improve the quality of life has continued its ever-accelerating pace in technology advancement. Device manufacturers continue their research and development efforts, for example, in improving the effectiveness, reliability, and safety of the devices, in mitigating the side effects resulting from device implantation, in improving ergonomic factors in deployment of the devices, and in reducing overall manufacturing costs. Patients and treating physicians are dissatisfied due to degradation in device performance as a result of gradual cell growth or accumulation (e.g., hyperplasia, restenosis, cell proliferation, formation of thrombus) on or near the surface of the implanted device. Such physiological reactions can adversely impact the devices, such impacts ranging from diminished effectiveness to complete malfunction.

Conventional approaches are available to mitigate such adverse cell growth. One approach is local administration of organic pharmacological agents or biological depressing agents that are known to have anticoagulant properties. Examples of such organic agents are heparin and other heparin complexes, such as heparin-tri(dodecyl)methylammonium chloride. The organic agents, typically mixed with a carrier (e.g., silane, siloxane coating), are coated on the surface or embedded in the subsurface of the device. Conventional coating techniques, such as dipping, spraying, painting, plasma deposition, solvent swelling, etc., may be used to attach the organic agents to the surface or the subsurface of the medical devices. A peripheral assembly or a separate delivery tool may be also required to deliver the organic agents to the surface of medical device during or after implantation. Another conventional approach to mitigate adverse cell growth on or around the medical device is local radiation. Use of low power lasers, thermal ablation, and radionuclides are examples of some techniques of choice. The following issued U.S. patents and published U.S patent applications, all incorporated herein by reference, are examples of these conventional approaches.

U.S. Pat. No. 5,665,077 issued to Rosen et al. describes drug treatment to prevent acute or sub-acute thrombic occlusion. A device is coated with a polymer mixed with the drugs, i.e., nitroso compounds.

U.S. Pat. No. 6,096,070 issued to Ragheb et al. describes a stent coated with at least one layer of a bioactive material (e.g., anitplatelet, antithrombotic, anti-inflammatory agents or other therapy agents) and one porous polymer layer posited over the bioactive material layer.

U.S. Pat. No. 6,106,454 issued to Berg et al. describes a medical device for localized delivery of radiation in vivo. The device has a porous structure where a water-insoluble radioactive salt is dispersed. The radioactive material can be loaded in the device just prior to implantation.

U.S. Pat. No. 6,179,789 issued to Tu et al. describes a stent comprising a coating of a radioactive substance. The same patent also describes an ablation system for delivering radiofrequency current to the stent for the purposes of thermally enhanced irradiation capability for tissue therapeutic treatment.

U.S. Pat. No. 6,238,872 issued to Mosseri describes a stent for treatment of restenosis. The stent is coated with an antigen. After the stent has been placed in the blood vessel, an antibody (i.e., a radioactive source) for treating restenosis is injected to bind the antigen on the stent.

U.S. Pat. No. 6,491,617 issued to Ogle et al. describes medical devices having a plurality of exogenous storage structures for storing a therapeutic agent (e.g., radioactive metal ions), which act to inhibit restenosis.

U.S. Patent Application Publication US2002/0042645 published for Shannon describes drug eluting stented tubular grafts wherein the stent has a coating comprising a composite of a polymer and a therapeutic substance.

U.S. Patent Application Publication US2003/0060877 published for Falotico et al. describes medical devices coated with therapeutic drugs to minimize or substantially eliminate a biological organism's reaction to the introduction of the medical device. Various materials and coating methodologies may be utilized to maintain the drugs on the device until delivered and positioned.

U.S. Patent Application Publication US2003/0088307 published for Shulze et al. describes a stent having a polymer coating. One or more bioactive agents (e.g., an anti-restenosis agent consisting of a potent analogue or derivative of tranilast) are disposed within the coating.

The above-mentioned conventional technologies, however, have their own drawbacks in application. For example, the organic agents are inherently sensitive to environmental effects, such as degradation from exposures of temperature, humidity, light, and chemicals. Local radiation is prone to equipment malfunction, operational error, need for multiple treatment, or inducement of other medical side effects. There is thus a need for innovative approaches offering alternative methods in suppressing or controlling cell growth on or near the surface of the implanted devices.

### SUMMARY OF THE INVENTION

The present invention relates to a pioneering approach of suppressing or controlling cell growth or accumulation on or near the surface of implanted medical devices. In particular, the surface of an implantable device is modified to comprise metal-containing compounds comprising metals in high oxidation states that, in turn, regulate proliferative cell growth on or near the surfaces of the device upon implantation.

The surface modification can be achieved by transforming one or more of the metals that already are present in the structural material of the device so that the surface of the device comprises one or more metal-containing compounds comprising one or more metals that are, at least in part, in a high oxidation state. Alternatively, the surface modification can be achieved by applying to the surfaces of the device one or more extrinsically formulated metal-containing compounds comprising one or more metals that are, at least in part, in a high oxidation state, and wherein the metal-containing compound may be applied to the surface of the medical device with or without the use of a binder, such as a polymer resin. Surface modification can also occur during or after implantation of the device by electrochemically forming metals in high oxidation states directly from the metals located on the surface of the device.

Additional coatings or layers can be applied to the modified surface of the device to protect the modified surface or to regulate the elution or release rate of the metal-containing compounds comprising metals in high oxidation states from the device. The eluted metal-containing compounds comprising metals in high oxidation states regulate proliferative diseases around the implanted medical devices.

### Definitions

High oxidation state metal-containing compounds or metal-containing compounds comprising metals in high oxidation states refer to compounds containing one or more of the following transition element metals present, at least in part, in the following oxidation states: chromium (IV), chromium (V), chromium (VI), manganese (V), manganese (VI), manganese (VII), cobalt (III) and nickel (III). The metals may be ionically or covalently bound to other elements in the metal-containing compounds. Compounds containing more than a natural distribution of radioactive isotopes are excluded from the definition of high oxidation state metal-containing compounds or metal-containing compounds comprising metals in high oxidation states.

Regulation of cell growth includes one or more of the following effects on proliferative tissue diseases: 1) cytostatic effects whereby cell division is either temporarily or permanently prevented, 2) cytotoxic effects whereby cells are induced to die either by necrosis, by apoptosis, or other cell death phenomena, and 3) anti-proliferative effects whereby cell migration or attachment is reduced or eliminated either as a direct effect on cells stimulated to migrate to or attach to a target tissue or by reducing a cell mediated process such as but not limited to inflammation that would induce cell migration or attachment.

The term “oxidation state” (a.k.a. oxidation number or valence) refers to the actual charge of a monatomic ion (e.g., Co²⁺, Cr³⁺, Cl⁻) or the hypothetical charge on an atom in a polyatomic group (e.g., SO₄²⁻, CrO₄²⁻, NO₃⁻, NH₄⁻) assigned by a set of standard rules. Oxidation states can be either positive or negative. Positive oxidation states indicate that the monatomic ion or the polyatomic group (hereinafter collectively “atom”) has become an “electron donor” in compound formation and thus is assumed to have “lost” a designated numbers of electrons to the nearby atoms. Negative oxidation states indicate that the atom has become an “electron acceptor” and is assumed to have “gained” a designated numbers of electrons. For example, Cl⁻ is assumed to have “gained” one electron, and O²⁻ is assumed to have “gained” two electrons from the adjacent atoms. In comparison, Pt²+is assumed to have “lost” two electrons, Cr³⁺ is assumed to have “lost” three electrons, and Cr⁶⁺ is assumed to have “lost” six electrons as a result of losing electrons to the adjacent atoms. The oxidation state is a particularly useful way to keep track of electrons in oxidation-reduction reactions.

“Transition metals” are elements in the midsection of the periodic table with atomic numbers 21-30, 39-48, 57-80, or 89-109. The transition metals show great similarities within a given period as well as within a given vertical group of the periodic table. Multiple oxidation states can be formed when transition metals form ionic compounds (a.k.a. “salts”) and covalent compounds with other elements. For example, chromium can form compounds wherein the chromium atom has different oxidation states: 2+ (e.g., CrBr₂), 3+ (e.g., Cr₂O₃, Cr(OH)₃), and 6+ (e.g., Na₂CrO₄, K₂Cr₂O₇). Thus, a Roman numeral or a description is typically used to indicate the positive oxidation state of a transition metal in such compounds (e.g., Cr(VI) or hexavalent chromium for Cr⁶⁺ in Na₂CrO₄ and K₂Cr₂O₇). The term “metal” or “transition metal” includes transition element metals in any oxidation state.

“Surface modification” in the present invention refers to providing high oxidation state metal-containing compounds on the surface of a medical device, wherein the compounds regulate proliferative cell growth on or near the surface of the implanted medical device.

### DETAILED DESCRIPTION OF THE INVENTION

The present invention reduces or eliminates undesirable cell growth or accumulation on or near the surface of an implanted medical device by providing high oxidation state metal-containing compounds on the surface of the device. The composition of these compounds comprise metals that are, at least in part, in a high oxidation state and that, in turn, cause cytostatic, cytotoxic, and anti-proliferative effects on the cells around the implanted medical devices.

Certain compounds containing transition metals are known to have cytotoxic effects when the metals are in high oxidation states. See e.g., Daryl E. Pritchard, et al., Mechanism of Apoptosis and Determination of Cellular Fate in Chromium(VI)-exposed Populations of Telomerase-immortalized Human Fibroblasts, Cell Growth & Differentiation, 12: 487-496, October 2001; Zhuo Zhang, et al. Cr(VI) Induces Cell Growth Arrest through Hydrogen Peroxide-Mediated Reactions, Molecular and Cellular Biochemistry, 222: 77-83, 2001; and Kirkwood A. Pritchard, Jr., et al., Chromium(VI) Increases Endothelial Cell Expression of ICAM-1 and Decreases Nitric Oxide Activity, Journal of Environmental Pathology, Toxicology and Oncology, 19(3):251-260, 2000. It is believed that the metal ions induce an oxidative stress that results in oxidative deterioration of cells; however, the mechanisms involved in the production of oxidative stress might be different. See Sidney J. Stohs, et al., Oxidative Mechanisms in the Toxicity of Chromium and Cadmium Ions, Journal of Environmental Pathology, Toxicology and Oncology, 20(2): 77-88, 2001. The present invention applies this seemingly undesirable property of certain transition metals in high oxidation states to suppress or control the growth and multiplication of cells on or near the surfaces of implanted medical devices.

One embodiment of the present invention involves modification of the surface of the medical device by producing compounds comprising metals in high oxidation states from the structural material (i.e., the metals) present in the medical devices themselves. In this embodiment, the medical device that undergoes surface modification is made of at least one alloy containing at least one transition metal, which when oxidized at least in part, can cause the desired regulation of proliferative tissue.

Transition metals are common elements in various alloys that are candidates for making medical devices. Device manufacturers choose specific alloys containing transition metals for various reasons. For instances, alloys having chromium, nickel, or cobalt can form oxides that adhere tightly to the metallic surface, protecting the metal from further oxidation or corrosion below the oxidized layer. Alloys 316L stainless steel (about 16-18% in chromium) and MP35N (about 19-21% in chromium) are widely used in the medical device industry partly for their unique property of corrosion resistance. Titanium and its alloys are popular candidates in applications demanding osteo-integration and biocompatibility. Nitinol (i.e., nickel-titanium alloy) “remembers” a preset shape after it is deformed; therefore, it can be made to return to its original shape at body temperature. Noble transition metals, e.g., gold, silver, platinum, and palladium, do not generally corrode even under harsh environments and also have good electrical conductivities. Conceivably, this embodiment of the present invention can be incorporated in numerous medical devices without adding extrinsic materials beyond the structural materials that are already present in the devices.

Chromium-containing alloys are widely used materials in the medical device industry; therefore, the various oxidation states of chromium will be used here to illustrate an embodiment of the invention. In accordance with this embodiment, higher oxidation states of the transition metal (e.g., Cr⁴⁺, Cr⁵⁺, or Cr⁶⁺) are produced from the base metal element (e.g., Cr) or from its lower oxidation states (e.g., Cr²⁺ or Cr³⁺).

As a transition metal, chromium can form polyvalent chromium ions such as Cr²⁺, Cr³⁺, Cr⁶⁺, and sometimes the intermediate states Cr⁴⁺ and Cr⁵⁺. In a typical ambient environment, the common forms of chromium ions are Cr²⁺ and Cr³⁺. For example, chromium in 316L stainless steel or MP35N can form chromium oxides (i.e., Cr²⁺ and Cr³⁺) on the surface of medical devices to prevent further oxidation below the oxide layer. In accordance with the present invention, elemental Cr and the naturally formed Cr²⁺ and Cr³⁺ ions on the device surfaces are oxidized, at least in part, to become Cr⁴⁺, Cr⁵⁺, and Cr⁶⁺. The transformation process from elemental Cr and its lower oxidation states (e.g., Cr²⁺ and Cr³⁺) to its higher states (e.g., Cr⁴⁺, Cr⁵⁺ and Cr⁶⁺) can be achieved by any conventional technologies that cause chromium atoms to lose electrons. Chemical or electrochemical reactions, light activation, heat activation, or combinations of these techniques are examples of technologies that could be used to produce transition metals such as chromium in high oxidation states.

The surfaces of medical devices generally are cleaned thoroughly to achieve the uniformity of transformation and to avoid interfering reactions.

The extent of surface modification (e.g., amounts and/or locations of Cr⁴⁺, Cr⁵⁺, and Cr⁶⁺) can be affected by factors such as the types of other elements present in the alloy, the percent chromium in the bulk material and/or on the surface, and the texture and grain configuration of the surface. The amount of Cr⁴⁺, Cr⁵⁺ or Cr⁶⁺ may vary with some factors including, but not limited to, the types of treatments (e.g., reduce or eliminate hyperplasia, restenosis, cell proliferation, formation of thrombus), the extent and longevity of the intended treatment, and the location of treatment.

In some instances, it may be unnecessary or even undesirable to modify the entire surface of the medical device. The location of surface modification can be selected to maximize the effectiveness with the least amount of Cr⁴⁺, Cr⁵⁺, and Cr⁶⁺. For example, in the situation of a stent, only the inside surface (i.e., blood contacting surface) may be modified to provide an effective treatment while the outside surface (i.e., lumen or muscle contacting surface) might be intentionally left unmodified. The growth of cells on the unmodified stent surface thus could enhance the fixation of the stent. In vitro or in vivo experiments or clinical trials can be used to validate or further optimize the configuration of surface modification for a particular medical device.

Laboratory testing (e.g., MEM Elution, Static Hemolysis) or in vivo testing on animals can be used to verify the biocompatibility of the modified devices in contact with the living cells. Biocompatible coatings can be applied to the modified surface of the medical device, which can protect the modified surface as well as regulate the elution rates of Cr⁴⁺, Cr⁵⁺, and Cr⁶⁺ for treatment. Processes for applying such coatings to medical devices are well known in the art, examples of which include, but are not limited to, dip coating, electrochemical deposition, vapor phase deposition processes such as plasma polymerization or paralene coating, and direct application of a preformed polymer film. See, for example, PCT Published Application WO 00/32255 to Klamath et al., incorporated herein by reference.

Conceivably, some applications may need higher levels of cytostatic, cytotoxic, or anti-proliferative effects for appropriate treatment. For example, an apparatus containing the present invention can be implanted to suppress cancer cells or tumors. Similar to the above descriptions, the elution rates of Cr⁴⁺, Cr⁵⁺ and Cr⁶⁺ can be regulated to obtain a particular result in treatment.

In another embodiment, the high oxidation state of the metals in the metal-containing compounds on the surface of the medical device might be generated from direct oxidation of surface metals and metal ions through application of electrochemical potentials. It is known, for example, that alloys containing chromium can have a potential applied to transform the oxidation state of Cr from Cr³⁺ to Cr⁶⁺. Such transformations could be done on the surface either during manufacturing, prior to implantation, or after implantation (i.e., in-situ) to generate the appropriate oxidation state of metals to induce the desired cytostatic, cytotoxic, or anti-proliferative effects.

Methods for preparing high oxidation state metal-containing compounds on or near the medical device surface, such as electrochemical oxidation or other wet-process oxidation such as hydrogen peroxide oxidation, can also be performed after applying an appropriate coating, such as a polymer coating, to the medical device. The high oxidation state metal-containing compounds may be formed underneath such a coating allowing for increased amounts of high-oxidation state metal-containing compounds at or near the medical device surface when aqueous or other wet chemical processes are used.

The following two examples (i.e., Examples 1 and 2) illustrate surface modification by way of oxygen plasma treatment on two alloy surfaces, i.e., 316L stainless steel and MP35N.

### EXAMPLE 1

Surface Modification of 316L Stainless Steel

In this example, the effect of O₂ plasma on the surface chemical composition of 316L stainless steel was evaluated. In particular, the amount of Cr⁶⁺ on the surface of the sample was investigated.

Five 316L stainless steel coupons were treated with O₂ plasma at various levels (i.e., Points 1-5 as denoted in Tables 1 and 2 below). Analyses of the five coupons were done using a Physical Electronics Quantum 2000 Scanning ESCA instrument with a monochromatic Al Kα x-ray source, an analysis area of 1400 micron² raster, a take-off angle of 45 degrees, and a charge correction of C—C, C—H in C Is spectra set to 284.8 eV.

The results of the elemental surface composition analysis of the five 316L coupons are shown in Table 1. The low amount of C (<20 at %) suggests that the surfaces of the coupons are fairly clean. The Fe to Cr ratio is ˜2 for all analysis points except for Point 2. This shows that the surface is enriched in Fe, significantly more so than typical electropolished/passivated 316L samples. The data indicate that the Point 2 sample has a different chemistry at the surface than do the other four, particularly in Fe and Cr content.

TABLE 1
Relative Atomic % Determined from ESCA Survey Spectra

| | at % | at % | at % | at % | at % | at % | at % | Cr/ |
|---|---|---|---|---|---|---|---|---|
| | C | O | P | Cr | Fe | Ni | Mo | Fe |
| 316L plasma pt | 16.54 | 60.28 | 1.4 | 6.3 | 12.37 | 2.6 | 0.52 | 0.509 |
| 1 | | | | | | | | |
| 316L plasma pt | 17.12 | 57.93 | 1.34 | 10.59 | 10.53 | 1.89 | 0.58 | 1.006 |
| 2 | | | | | | | | |
| 316L plasma pt | 17.13 | 59.19 | 0.56 | 6.2 | 13.21 | 3.14 | 0.56 | 0.469 |
| 3 | | | | | | | | |
| 316L plasma pt | 16.87 | 59.88 | 0.56 | 5.85 | 13.22 | 3.09 | 0.52 | 0.442 |
| 4 | | | | | | | | |
| 316L plasma pt | 17.65 | 59.65 | nd | 6.22 | 13.03 | 2.91 | 0.55 | 0.477 |
| 5 | | | | | | | | |

The oxide was evaluated by high-resolution XPS spectroscopy. Cr⁶⁺ was identified in all of the samples (see Table 2). The amount of Cr⁶⁺ was about 40% for all of the samples (relative to Cr²⁺ and Cr³⁺), except for sample 2, which showed significantly less Cr⁶⁺ (˜10%). In comparison, samples of untreated 316 L stainless steel typically have Cr⁶⁺/Cr^{2+/3+} ratios of 0.04 +/−0.002.

TABLE 2
Relative amount of Cr species

| | Cr^{2+/3+} | Cr⁶⁺ | Cr⁶⁺/Cr^{2+/3+} |
|---|---|---|---|
| 316L plasma pt 1 | 0.63 | 0.37 | 0.587 |
| 316L plasma pt 2 | 0.90 | 0.10 | 0.111 |
| 316L plasma pt 3 | 0.54 | 0.46 | 0.852 |
| 316L plasma pt 4 | 0.55 | 0.45 | 0.818 |
| 316L plasma pt 5 | 0.60 | 0.40 | 0.667 |

For the other metal species (Co, Mo, Ni), high resolution spectroscopy was done as well. The data show them to be present mainly in the oxide state. There is a small signal for the non-oxidized metal component for all species.

### EXAMPLE 2

Surface Modification of MP35N

In this example, the effect of O₂ plasma on the surface chemical composition of MP35N was evaluated. In particular, the amount of Cr⁶⁺ on the surface of the sample was investigated.

Six MP35N coupons were treated with O₂ plasma at various levels (i.e., Points 1-6 as denoted in Tables 1 and 2 below). Analyses of the six coupons (2 points/coupon analyzed) were done using a Physical Electronics Quantum 2000 Scanning ESCA instrument with a monochromatic Al Kα x-ray source, an analysis area of 1400 micron² raster, a take-off angle of 45 degrees, and a charge correction of C—C, C—H in C Is spectra set to 284.8 eV.

The results of the elemental surface composition of the MP35N coupons are shown in Table 3. The low amount of C (<20 at %) suggests that the surfaces are fairly clean. The Cr/Co ratio is 0.63-0.85, which compares to values of 2.9-7.0 for MP35N that has been similarly prepared but not O₂ plasma treated. The O₂ plasma does not seem to have enhanced the Cr concentration.

TABLE 3
Relative Atomic % Determined from ESCA Survey Spectra

| | at % | at % | at % | at % | at % | at % | |
|---|---|---|---|---|---|---|---|
| | C | O | Cr | Co | Ni | Mo | Cr/Co |
| MP35N pt 1 | 17.49 | 53.77 | 6.65 | 10.27 | 10.28 | 1.55 | 0.648 |
| MP35N pt 2 | 16.93 | 54.09 | 6.51 | 10.28 | 10.78 | 1.42 | 0.633 |
| MP35N pt 3 | 17.03 | 53.24 | 7.27 | 10.66 | 10.16 | 1.64 | 0.682 |
| MP35N pt 4 | 18.3 | 55.13 | 7.68 | 9 | 8.17 | 1.72 | 0.853 |
| MP35N pt 5 | 19.8 | 53.37 | 7.27 | 9.62 | 8.46 | 1.48 | 0.756 |
| MP3SN pt 6 | 29.89 | 47.05 | 6.45 | 7.74 | 7.4 | 1.47 | 0.833 |

The oxide was evaluated by high-resolution XPS spectroscopy. Cr⁶⁺ was identified in all of the samples (see Table 4). The amount of Cr⁶⁺ ranged from 27 to 58% for all samples (relative to Cr²⁺ and Cr³⁺). This is about the same amount of Cr⁶⁺/Cr^{2+/3+} as found in the 316L samples of Example 1. In comparison, samples of untreated MP35N typically have Cr⁶⁺/Cr^{2+/3+} ratios of 0.03+/−0.005.

TABLE 4
Relative amount of Cr species

| | Cr^{2+/3+} | Cr⁶⁺ | Cr⁶⁺/Cr^{2+/3+} |
|---|---|---|---|
| MP35N pt 1 | 0.50 | 0.50 | 1 |
| MP35N pt 2 | 0.43 | 0.57 | 1.326 |
| MP35N pt 3 | 0.42 | 0.58 | 1.381 |
| MP35N pt 4 | 0.73 | 0.27 | 0.371 |
| MP35N pt 5 | 0.55 | 0.45 | 0.818 |
| MP35N pt 6 | 0.65 | 0.35 | 0.538 |

For the other metal species (Co, Mo, Ni), high-resolution spectroscopy was done as well. The data show them to be present mainly in the oxide state, although there is a small signal for the non-oxidized metal component for all species.

In a further embodiment, the approach of modifying the surface of a medical device so that the surface comprises metal-containing compounds comprising metals in high oxidation states can also be achieved by applying extrinsic compounds to the surface of medical devices. In accordance with the present invention, such compounds can be produced extrinsically and subsequently applied (with or without binders, such as polymer resins) to the surface or the subsurface of medical devices. As with the previous embodiments discussed, the high-oxidation state metal-containing compounds are subsequently eluted from the inorganic compounds after implantation of the device to produce the desired cytostatic, cytotoxic, or anti-proliferative effect. And as with the previous embodiments discussed, coatings or other inhibiting means can be applied to the modified surface of the medical device, which can protect the modified surface as well as regulate the elution rates of the high oxidation state metal-containing compounds.

For illustrative purposes of this embodiment, compounds such as Na₂CrO₄ or K₂Cr₂O₇ (the chromium ions in both salts are Cr⁶⁺) are produced in a controlled environment. The compounds then can be applied to the surface of the device with or without being mixed with a binder(s), such as a polymer resin. Using polymer resins are advantageous because they can dilute the concentration of the compounds, improve the uniformity of the distribution of the compounds, improve surface adhesion and loading of the compounds, and regulate elution rates of the compounds. Cationic polymers, such as polyethyleneimine, polyethylene glycol, and polyvinylpyrolidone with polyethyleneimine, are examples of good candidates for binding negatively charged groups such as CrO₄²⁻ (i.e., chromate ion) and Cr₂O₇²⁻ (i.e., dichromate ion) to surfaces, although many cationic polymers could be effective.

Example 3 illustrates a mixture of compounds containing metals in high oxidation states (e.g., Cr⁶⁺) that are produced in a laboratory and then subsequently applied to a 31 6L stainless steel substrate. Example 4 illustrates the anti-proliferative character (i.e., dose dependent effects of Cr⁶⁺ on proliferation of human coronary artery smooth muscle cells) of a high oxidation state metal-containing compound (i.e., potassium chromate) in accordance with this invention.

### EXAMPLE 3

Cr⁶⁺ Compounds Applied to 316L Stainless Steel Coupons

For this example, a polymer with the following composition (see Table 5) was synthesized.

TABLE 5

| Component | Molar Ratio | Weight % |
|---|---|---|
| BMA | 65 | 53.08 |
| M40G | 20 | 32.78 |
| AEMH | 15 | 14.14 |
| Totals | 100 | 100.00 |

BMA = n-butyl methacrylate: 142 g/mole
M40G = methoxy polyethylene glycol 230 methacrylate: 285 g/mole
AEMH = amino ethyl methacrylate hydrochloride: 164.2 g/mole

The polymer was then dissolved in a mixture of solvent containing 1:1 chloroform and methanol. The polymer solution was then saturated with either sodium chromate or dichromate. Since exact solubility of the chromate salts in the polymer solution was not known, and based on the fact that the solutions were saturated, it was necessary to let the solution settle for a short time to avoid getting large granules of salt on the surface of dipped coupons as described below.

Twenty 316L stainless steel coupons were ultrasonically cleaned in isopropyl alcohol for 5 minutes and then air-dried. Each coupon was dipped in the polymer solution, then suspended with the coated side down and allowed to dry overnight. The dried coupons were soaked for specific time periods in deionized water to elute chromate or dichromate ions. Table 6 shows the schedule of soaking times for the coupons.

TABLE 6

| Coupon # | W (cm) | L (cm) | tₚᵣₑ (μm) | tₚₒₛₜ (μm) | Δt (cm) | V (dm³) | Solution Composition | Soak Time |
|---|---|---|---|---|---|---|---|---|
| 1 | 1.27 | 1.42 | 2.27 | 2.30 | 3.00E−05 | 5.4102E−08 | Na₂CrO₄ + 300 K | 1 min |
| 2 | 1.26 | 1.75 | 22.9 | 22.5 | 6.00E−05 | 1.3230E−07 | Na₂CrO₄ + 1 Mil | 1 min |
| 3 | 1.25 | 1.49 | 22.7 | 23.0 | 3.006−05 | 0.59756−08 | Na₂CrO₄ + 300 K | 1 hour |
| 4 | 1.25 | 1.72 | 22.5 | 23.5 | 1.006−04 | 2.15006−07 | Na₂CrO₄ + 1 Mil | 1 hour |
| 5 | 1.27 | 1.50 | 22.0 | 23.0 | 5.006−05 | 9.5250E−08 | Na₂Cr₂O₇ + 300 K | 1 min |
| 6 | 1.25 | 1.84 | 22.5 | 23.7 | 1.206−04 | 2.76006−07 | Na₂Cr₂O₇ + 1 Mil | 1 min |
| 7 | 1.26 | 1.51 | 22.9 | 23.1 | 2.00E−05 | 3.90526−05 | Na₂Cr₂O₇ + 300 K | 1 hour |
| 8 | 1.20 | 1.63 | 22.9 | 23.7 | 8.00E−05 | 1.8300E−07 | Na₂Cr₂O₇ + 1 Mil | 1 min |
| 9 | 1.26 | 1.42 | 22.7 | 23.0 | 3.006−05 | 5.3676E−06 | Na₂CrO₄ + 300 K | 1 day |
| 10 | 1.27 | 1.74 | 22.9 | 23.9 | 1.006−04 | 2.20966−07 | Na₂CrO₄ + 1 Mil | 1 day |
| 11 | 1.26 | 1.49 | 22.9 | 23.1 | 2.006−05 | 3.75400−00 | Na₂CrO₄ + 300 K | 1 week |
| 12 | 1.28 | 1.75 | 22.9 | 26.7 | 3.800−04 | 8.51200−07 | Na₂CrO₄ + 1 Mil | 1 week |
| 13 | 1.27 | 1.50 | 22.9 | 23.1 | 2.006−05 | 3.81000−00 | Na₂Cr₂O₇ + 300 K | 1 day |
| 14 | 1.26 | 1.01 | 22.9 | 24.0 | 1.106−04 | 2.55870−07 | Na₂Cr₂O₇ + 1 Mil | 1 day |
| 15 | 1.26 | 1.50 | 22.5 | 23.0 | 5.006−05 | 9.60006−08 | Na₂Cr₂O₇ + 300 K | 1 week |
| 16 | 1.26 | 1.80 | 22.9 | 23.5 | 6.006−05 | 1.36060−07 | Na₂Cr₂O₇ + 1 Mil | 1 week |
| 17 | 1.26 | 1.49 | 22.7 | 23.0 | 3.000−05 | 5.63220−06 | Na₂CrO₄ + 300 K | 2 weeks |
| 16 | 1.25 | 1.74 | 22.5 | 23.5 | 1.006−04 | 2.17500−07 | Na₂CrO₄ + 1 Mil | 2 weeks |
| 19 | 1.26 | 1.51 | 22.7 | 23.0 | 3.000−05 | 5.79646−90 | Na₂Cr₂O₇ + 300 K | 2 weeks |
| 20 | 1.25 | 1.80 | 22.7 | 23.9 | 1.200−04 | 2.70000−07 | Na₂Cr₂O₇ + 1 Mil | 2 weeks |

Definitions of column headings:
W = width of coupon = polymer coating
L = length of polymer coating adhered to coupon
tₚᵣₑ = thickness of coupon, pre−dip
tₚₒₛₜ = thickness of coupon = polymer, post−dip
Δt = thickness of polymer coating = tₚₒₛₜ − tₚᵣₑ
V = volume of polymer deposited on metal surface

UV-Vis absorbance data was gathered on a Hewlett Packard 8452A Diode Array Spectrophotometer. From the UV-Vis absorbance data results, the ampunts of Cr⁶⁺ were calculated by a simple ratio of the unknown sample with a standard. The calculation was performed using the formula: [standard]/A_{std.}=[X]/Aₓ. Table 7 shows the calculated elution rates of Cr^{6+.}

TABLE 7
Determination of Concentration for Samples #1-20

| λₘₐₓ chromate ion = | | 372 nm | | | |
|---|---|---|---|---|---|
| Concentration of Standard K₂CrO₄ Solutions: | | | 1.0E−05 | 1.0E−04 | |
| Absorbance of Standard K₂CrO₄ Solutions: | | | 2.0844E−02 | 0.41391 | |
| Coupon # | Absorbance | Concentration 1* | Concentration 2** | Ave. Conc. | Detection† |
| 1 | −3.6621E−03 | −1.7569E−06 | −8.8476E−07 | −1.3208E−06 | No |
| 2 | −6.5613E−03 | −3.1476E−06 | −1.5652E−06 | −2.3665E−06 | No |
| 3 | −8.3466E−03 | −4.0043E−06 | −2.0165E−06 | −3.0104E−06 | No |
| 4 | −8.2397E−03 | −3.9530E−06 | −1.9907E−06 | −2.9719E−06 | No |
| 5 | −9.1705E−03 | −4.3996E−06 | −2.2156E−06 | −3.3076E−06 | No |
| 6 | −8.4381E−03 | −4.0462E−06 | −2.0386E−06 | −3.0434E−06 | No |
| 7 | −1.3580E−03 | −6.5151E−07 | −3.2809E−07 | −4.8980E−07 | Yes |
| 8 | −5.8136E−03 | −2.7891E−06 | −1.4046E−06 | −2.0968E−06 | No |
| 9 | 1.0529E−03 | 5.0513E−07 | 2.5438E−07 | 3.7976E−07 | Yes |
| 10 | −1.1230E−02 | −5.3876E−06 | −2.7132E−06 | −4.0504E−06 | No |
| 11 | 4.4098E−03 | 2.1156E−06 | 1.0654E−06 | 1.5905E−06 | Yea |
| 12 | −1.0544E−02 | −5.0585E−06 | −2.5474E−06 | −3.8030E−06 | No |
| 13 | 4.9637E−02 | 2.3814E−05 | 1.1992E−05 | 1.7903E−05 | Yes |
| 14 | 3.7079E−03 | 1.7789E−06 | 8.9582E−07 | 1.3374E−06 | Yes |
| 15 | 8.4274E−02 | 4.0431E−05 | 2.0360E−05 | 3.0396E−05 | Yes |
| 16 | 9.5520E−02 | 4.5826E−05 | 2.3077E−05 | 3.4452E−05 | Yes |
| 17 | 3.6774E−03 | 1.7642E−06 | 8.8845E−07 | 1.3264E−06 | Yes |
| 18 | −5.2795E−03 | −2.5329E−06 | −1.2755E−06 | −1.9042E−06 | No |
| 19 | 1.2387E−01 | 5.9427E−05 | 2.9927E−05 | 4.4677E−05 | Yes |
| 20 | 9.0729E−02 | 4.3528E−05 | 2.1920E−05 | 3.2724E−05 | Yes |

*Concentration 1 calculated based on the (1.0E−05)M standard
**Concentration 2 calculated based on the (1.0E−04)M standard
†Detection was based on whether or not uv−vis plot showed absorbance peak

### EXAMPLE 4

The Effect of Potassium Chromate (Containing Cr⁶⁺) on Human Coronary Artery Smooth Muscle Cell (“HCASMC”) Proliferation After a 3-day Exposure

The CellTiter-Glo Luminescent Cell Viability Assay from Promega is a method of determining the number of viable cells in culture based on the quantities of ATP present, which signals the presence of metabolically active cells. A single reagent is added to cells directly in culture. This reagent lyses the cells and provides the luciferase enzyme that reacts with the liberated ATP to create the luminescent signal.

On Day 0, a 24-well plate was seeded with 5×10³ HCASMC per well (n=3). Cells were placed in the incubator for 6 hrs before potassium chromate was added to allow for proper adherence. 1 M potassium chromate stock solution was made in serum-free media and dilutions were made to study the following range: 5, 1, 0.5, 0.1, 0.05, 0.01, 0.005, 0 μM. The final volume in each well was 1 mL. On Day 3, the proliferation assay was performed. The results of this experiment are shown in the following two graphs.

[image]

The morphology of SMC growing in the presence of potassium chromate is indicative of cell distress. Dose-dependent cytostatic phenomenon was observed, while cells in the control condition (O M) showed good adhesion and spreading. In comparison, cells in 5 μM potassium chromate showed rounded morphology, likely with poor adhesion. In addition, the cells exposed to 5 ,μM, 1 μM and 0.5 μM potassium chromate did not begin to round up until 24 to 48 hours after exposure. On the basis of previous experience, cells exposed to potent cytotoxic compounds would typically round up within the first 12 hours.

The high oxidation state metal-containing compounds can be applied selectively on particular surfaces of medical devices. Examples of metal deposition technology generally can be found, for example, in U.S. Pat. No. 6,254,635 issued to Schroeder et al., which is incorporated herein by reference. Other metal deposition methods may also be available as technology continues to advance.

Other transition metals (e.g., silver, platinum, palladium, nickel, cobalt, titanium) can also be used for suppressing cell growth on or near the implanted medical device. Like chromium, those transition metals are capable of or prone to form various compounds with multiple oxidation states. For examples: cobalt can form the ions Co²⁺ and Co³⁺, titanium can form the ions Ti²⁺, Ti³⁺, and Ti⁴⁺, manganese can form the ions Mn²⁺, Mn³⁺, Mn⁴⁺, Mn⁵⁺, Mn⁶⁺, and Mn⁷⁺, and vanadium can form the ions V²⁺, V³⁺, V⁴⁺, and V⁵⁺. Similar to chromium, those transition metal ions and their compounds can also cause metabolic effects similar to those of Cr⁶⁺ in controlling the cell growth or accumulation. See Zhuo Zhang, et. al. Cr(VI) Induces Cell Growth Arrest through Hydrogen Peroxide-Mediated Reactions, Molecular and Cellular Biochemistry 222: 77-83, 2001 (discussion of Cr⁶⁺ toxicity).

The description of the invention is intended to be illustrative. Other embodiments, modification and equivalents may be apparent to those skilled in the art without departing from its spirit.

We claim:

1. A medical device for treating proliferative disorders in a mammal, comprising:
a structural member;
a high oxidation state metal-containing compound on or near the surface of the structural member wherein the metal-containing compound comprises a metal selected from the group consisting of chromium (IV), chromium (V), chromium (VI), manganese (V), manganese (VI), manganese (VII), cobalt (III), nickel (III) and combinations thereof; and
a polymeric layer disposed over at least part of the structural member and the metal-containing compound, wherein the polymeric layer regulates the release rate of the metal-containing compound from the medical device upon implantation of the medical device in the mammal.

2. The medical device of claim 1, wherein the metal is selected from the group consisting of chromium (IV), chromium (V), chromium (VI) and combinations thereof.

3. The medical device of claim 1, wherein the metal is selected from the group consisting of manganese (V), manganese (VI), manganese (VII) and combinations thereof.

4. The medical device of claim 1, wherein the structural member is a stent.

5. The medical device of claim 1, wherein the high oxidation state metal-containing compound is substantially non-radioactive.

6. A method of treating a proliferative disorder in a mammal, comprising:
implanting into the mammal a medical device at or near the site of the proliferative disorder, wherein the medical device comprises a structural member and a high oxidation state metal-containing compound on or near the surface of the structural member, and wherein the metal containing compound comprises a metal selected from the group consisting of chromium (IV), chromium (V), chromium (VI), manganese (V), manganese (VI), manganese (VII), cobalt (III), nickel (III) and combinations thereof; and
releasing in the mammal a therapeutically effective amount of the metal-containing compound from the medical device, wherein the metal-containing compound regulates cell growth.

7. The method of claim 6, wherein the metal is selected from the group consisting of chromium (IV), chromium (V), chromium (VI), and combinations thereof.

8. The method of claim 6, wherein the metal is selected from the group consisting of manganese (V), manganese (VI), manganese (VII), and combinations thereof.

9. The method of claim 6, wherein the proliferative disorder is a vascular disorder.

10. The method of claim 6, wherein the proliferative disorder is a cancer.

11. The method of claim 6, wherein the medical device additionally comprises a polymeric layer disposed over at least part of the structural member and the metal-containing compound, thereby affecting the release of the metal-containing compound into the mammal.

12. The method of claim 6, wherein the high oxidation state metal-containing compound is substantially non-radioactive.
